(19) United States
(12) Patent Application Publication
(10) Pub. No.: US 2004/0220089 A1
(43) Pub. Date: Nov. 4, 2004

(54) Ophthalmic preparation containing glycoprotein

(76) Inventors: Edward J. Ellis, Lynnfield, MA (US); Charles D. Leahy, Concord, MA (US); Jeanne Y. Ellis, Lynnfield, MA (US)

Correspondence Address: CANTOR COLBURN, LLP, 55 GRIFFIN ROAD SOUTH, BLOOMFIELD, CT, 06002

(21) Appl. No.: 10/814,001

(22) Filed: Mar. 30, 2004

Related U.S. Application Data
(63) Continuation-in-part of application No. PCT/US02/31657, filed on Oct. 3, 2002.
(60) Provisional application No. 60/326,912, filed on Oct. 3, 2001.

(51) Int. Cl. A61K 38/38

(52) U.S. Cl. 514/8

(57) ABSTRACT

The present invention relates to an aqueous formulation to be instilled into the eye, or in which to pre soak or store an object to be inserted into the eye, such as a contact lens, an ointment, or a solid device to be inserted into the conjunctival sac. The preparations disclosed are utilized for the treatment of a tear film and ocular surface disorder known as keratoconjunctivitis sicca or dry eye syndrome. In general, the preparations of this invention are also effective for the relief of symptoms of eye irritation, such as those caused by dry environmental conditions or by contact lens wear. In accordance with the present invention, the ophthalmic preparation includes a glycoprotein component, similar to that found at the normal human ocular surface and in one exemplary and preferred embodiment, the glycoproteins are derived from mammalian milk, preferably bovine.

### CROSS-REFERENCE TO RELATED APPLICATIONS

This application is a continuation-in-part of International patent application No. PCT/US02/31657, filed Oct. 3, 2002, which claims benefit to U.S. provisional patent application No. 60/326,912, filed Oct. 3, 2001, both of which are hereby incorporated by reference in their entirety. Additionally, this application is related to pending U.S. patent application entitled, “OPHTHALMIC PREPARATION CONTAINING GLYCOMACROPEPTIDE”, filed by Applicant on Mar. 30, 2004, which is herein incorporated by reference in its entirety.

### STATEMENT REGARDING FEDERAL SPONSORSHIP

The U.S. Government has a paid-up license in this invention and the right in limited circumstances to require the patent owner to license others on reasonable terms as provided for by the terms of Grant Nos. 1 R43 EY12573-01, 2R44EY12573-02 and 5R44EY12573-03 awarded by the National Institute of Health.

### TECHNICAL FIELD

The present invention relates to ophthalmic preparations and more specifically relates to ophthalmic preparations for use as a tear film supplement, wherein the preparation includes at least one of a glycoprotein component.

### BACKGROUND

Initial descriptions and models of the tear film described the tear film as including three distinct layers and as being a three-layered, aqueous-dominated tear film. One of the layers comprises a mucin layer which serves primarily to render the hydrophobic ocular surface hydrophilic, so that the aqueous layer comprising the bulk of the tear film will spread evenly over the eye.

Current work in this field has shown that the classic aqueous-dominated tear film model has been replaced by the more probable concept of a mucin-dominated gel. This gel has its highest concentration of mucin at the epithelial surfaces of the cornea and conjunctiva, and the mucin concentration gradually decreases farther out into the tear film. In this model, the presence of mucin remains significant for the structure, stability and function of the entire tear film. Recent studies of the tear film using laser interferometry and confocal microscopy might be including the entire gel layer in indicating that the human tear film is 30 to 40 microns thick, more than four times thicker than earlier estimates.

Based on tear film physiology and clinical observations, tear film abnormalities are commonly designated by focus on a specific deficiency, such as an aqueous tear deficiency, kerato-conjunctivitis sicca (KCS), a mucin deficiency, a lipid abnormality, an impaired lid function, or an epitheliopathy. Although clinically useful, the simplistic concept of a lack of one component of the tear film as the cause of dry eye has given way to a much more sophisticated view of ocular surface disease that involves: (1) the health and regulation of the various glands contributing secretions to the tear film, (2) changes in the tear film itself, such as in osmolality and content of inflammatory mediators, and (3) what is viewed as a sort of “final common pathway”, the subsequent changes to the ocular surface. In fact, many clinicians and authors prefer the term “ocular surface disease” over “dry eye”, for it is change to the ocular surface, whatever the original cause, that results in the significant signs and symptoms of dry eye. The discomfort of ocular surface disease is expressed in ocular symptoms, such as dryness, grittiness, burning, soreness or scratchiness, with variation among individuals. These symptoms can also be exacerbated by factors such as environmental conditions, computer operation and contact lens wear. The combination of varying clinical signs and symptoms has also been termed dry eye syndrome.

Over the past twenty to thirty years many attempts have been made to provide an effective and long lasting treatment of dry eye symptoms, particularly for patients with moderate to severe KCS. These prior art attempts can be categorized on the basis of their physical state: ointments, emulsions, solid devices and aqueous based solutions or gels.

Ointments are generally anhydrous preparations based on mixtures of white petrolatum and mineral oil. Because these formulations are greasy and cause blurred vision, they are not widely used other than in cases of severe symptoms, and are mostly limited to application at night just before sleeping. Emulsion based formulations for treating dry eye symptoms have emerged over the past ten years. One approach has been disclosed in a series of U.S. Pat. Nos.: 5,578,586; 5,371,108; 5,294,607; 5,278,151; 4,914,088, all of which are herein incorporated by reference in their entirety. These patents teach the methods and compositions for reducing evaporation of the aqueous layer from the surface of the eye. The method comprises applying an admixture of a charged phospholipid and a non-polar oil over the eye, preferably in the form of a finely divided oil-in-water emulsion. Another approach is described in U.S. Pat. Nos. 4,818,537 and 4,804,539, incorporated herein by reference in their entirety, where liposome compositions in the form of emulsions are claimed to provide enhanced retention on ocular surfaces and thereby alleviate the symptoms of dry eye.

Solid devices, in the form of ocular inserts, have been utilized for longer term symptomatic relief of dry eye. These devices are placed in the eye and slowly dissolve or erode to provide a thickened tear film. Often patients find these devices difficult to insert and once in place, they tend to be uncomfortable.

The most recommended and commercially successful methodology to treat dry eye symptoms is aqueous based solutions or gels. For the patient, eye drops are convenient and easy to apply relative to the other options mentioned above. There are at least thirty artificial tear products currently on the market from which to choose. For the most part the “active” ingredients in these present day artificial tear formulations are common water soluble or dispersable polymers such as: hydroxyethylcellulose; hydroxypropylmethylcellulose; methylcellulose; carboxymethylcellulose; polyvinyl alcohol; polyvinyl pyrrolidone; polyethylene glycol; carbomers; and poloxamers.

These currently marketed products, while providing temporary relief of symptoms—usually measured in minutes—are strictly palliative without long term effect. In fact, to truly maintain relief of symptoms in moderate to severe cases, an impractical schedule of doses would be necessary. With preserved solutions, the frequency of instillation can lead to signs and symptoms of irritation, making it necessary to utilize expensive and more cumbersome unit dose delivery packages.

The recent patent literature indicates a continued interest in pursuing synthetic based artificial tear solutions. For example, U.S. Pat. No. 5,460,834, incorporated herein by reference in its entirety, teaches the use of hydroxypropylmethylcellulose along with other ingredients as an ophthalmic solution, and U.S. Pat. No. 6,180,093 incorporated herein by reference in its entirety, discloses the use of polyvinylpyrrolidone in combination with other components to relieve eye dryness.

The art recognizes that an ophthalmic solution must provide an effective and long lasting treatment for symptoms of dry eye. One approach to achieving these aims is to provide a solution with tailored rheological properties, that is, a high viscosity solution that yields or flows under stress. Examples of this approach are disclosed in U.S. Pat. Nos. 5,075,104 and 5,209,927, incorporated herein by reference in their entirety, where the rheological properties of the ophthalmic solutions are attained through the use of carbomer polymers. These carbomer polymers have been found to be bio-adhesive as described in U.S. Pat. Nos. 5,225,196, 4,983,392 and 4,615,697, all of which are incorporated by reference in their entirety. It is believed that the bio-adhesive properties of the carbomer contributes to longer retention times in the eye. In fact, U.S. Pat. Nos. 5,075,104 and 5,209,927, incorporated by reference in their entirety, teach “that the carbomer polymers appear to function by maintaining or restoring the normal hydration equilibrium of the epithelial cells, thus protecting the cornea.

The search for useful ophthalmic solution polymers has extended into the area of bio polymers, with particular emphasis on the naturally occurring polysaccharides. One polymer, hyaluronic acid, and its sodium salt have received much attention over the past several years. In fact, one commercial product, Hylashield®, based on a high molecular weight sodium hyaluronate, has been successfully marketed as a dry eye treatment solution. The use of hyaluronic acid in artificial tear solution compositions is also taught in U.S. Pat. No. 5,460,834 which is incorporated by reference in their entirety. Other polysaccharides, such as carrageenan, tamarind gum and keratan sulfate have been claimed to have utility in artificial tear solutions as disclosed in U.S. Pat. Nos. 5,403,841 and 5,460,834, and 6,056,950, all of which are incorporated by reference in their entirety. In addition, polysaccharides, such as alginate, dextran, scleroglucan and xanthan have been used, or have been proposed for use in ophthalmic solutions.

The patent literature reveals one dated reference to the use of mucin in sterilized, preserved and stable solutions. U.S. Pat. No. 4,438,100, incorporated herein by reference in its entirety, describes mucin-containing solutions for application to sensitive mucous membranes of the oral cavity, the nasal system and the eye. The mucins utilized in this invention are non human mammalian mucins selected from the group consisting of buccal and gastrointestinal mucins. In fact, the source of their mucins is mucus, a mature and complex secretion containing a mixture of various mucin molecules as well as other proteins and associated contaminants of secretion. There is no distinction made between secreted mucins and mucins expressed by the surface cells of the oral cavity or gastrointestinal mucous membranes. Two very recent publications, U.S. Pat. No. 6,281,192 and U.S. Pat. No. 6,429,194, incorporated by reference in their entirety disclose ophthalmic applications of mucin derived from mammalian milk or milk byproducts: the mucin described was found to be a MUC1 type mucin similar to the transmembrane mucin expressed on the surface of the human eye. The presence of a small quantity of mucin in milk and has been the subject of a review article; Patton, S. Gendler, S. J., and Spicer, A. P., “The Epithelial Mucin, MUC1, of Milk, Mammary Gland and Other Tissues,” Biochemica et Biophysics Acta 1241 (1995) 407-424. This is not unexpected since mucin is an integral membrane component. Recently, mucin has been identified as a minor component in dairy whey. More recently, mucin, MUC1, was isolated and purified from bovine milk fat globule membranes. The extensive characterization of this recovered MUC1 was reported in a published article: Pallesen, L. T., Andersen, M. H., Nielsen, R. L., Berglund, L., Petersen, T. E., Rasmussen, L. K., and Rasmussen, J. T., “Purification of MUC1 from Bovine Milk-Fat Globules and Characterization of a Corresponding Full-Length cDNA Clone,” Journal of Dairy Science Vol 84, No 12 (2001) 2591-2598.

### SUMMARY

The present application relates to ophthalmic preparations for use as a tear film supplement. More specifically, the invention relates to an ophthalmic formulation to be instilled into the eye, or in which to pre treat or store an object to be inserted into the eye, such as a contact lens or a solid device to be inserted into the conjunctival sac. The preparations disclosed are utilized for the treatment of disorders such as keratoconjunctivitis sicca or dry eye syndrome. In general, the preparations of this invention are also effective for the relief of symptoms of eye irritation, such as those caused by dry environmental conditions or by contact lens wear.

In particular, the present application relates to ophthalmic compositions including at least one milk-derived glycoprotein component, as well as to methods for their preparation and usage. The application also relates to a method of treating the eye by topically applying the composition of the present invention, when indicated, to provide lubrication and protection of the ocular surface, for the relief of dryness and discomfort symptoms, such as experienced in patients with dry eye and following traumatic injury or surgery, and when indicated to achieve the other effects mentioned above. In one preferred embodiment the compositions of the present invention are provided as buffered, sterile aqueous solutions. The subject compositions may be unpreserved (provided in a unit dose format) or may be preserved(multi dose format). These compositions are supplied in pharmaceutical containers and with appropriate labeling and instructions for use.

In one preferred embodiment, the glycoproteins are isolated from mammalian milk or milk byproducts. In another preferred embodiment the glycoproteins are isolated from bovine milk. In yet another preferred embodiment the glycoproteins are derived from dairy whey, a byproduct of cheese making. To form the ophthalmic preparations disclosed herein, other ingredients commonly employed in ophthalmic formulations are utilized to provide a balance of physiologically acceptable properties, depending on whether the final product is a solution, ointment, gel, lotion or solid. However, it will be understood that the glycoproteins can be derived from a number of other sources so long as the materials are suitable for the intended use described herein.

The above-discussed and other features and advantages of the present invention will be appreciated and understood by those skilled in the art from the following detailed description.

### DETAILED DESCRIPTION OF THE PREFERRED EMBODIMENTS

Glycoproteins are of great interest as components of mammalian membranes. Structurally they are composed of relatively short carbohydrate sequences covalently linked to a protein core. The glycoproteins of the cell surface appear to function in recognition, intercellular adhesion and modulation of certain intracellular transport event. That one characteristic phenotypic expression of tumorigenic transformation is an alteration in cell surface glycoproteins strongly implies an involvement of glycoproteins in at least several cell surface functions. It is believed that the glycoproteins of this invention have biological activity. They may enhance the production and stability of the tear film, and in particular may enhance the interaction of the aqueous tears and ocular surface glycoproteins, mucins and mucus. Physical activity and mechanisms of interaction of glycoproteins also contribute to protection of the surface. The carbohydrate groups of the protein are hydrophilic and have the ability to hold water and enhance the aqueous environment at the ocular epithelial cellular surface. The glycoproteins provide a physical mechanism of protection by their presence, bulk and hydration, as well as by binding pathogens and debris in their carbohydrate side chains.

Lipid globules in milk are enclosed in a membrane which is derived directly from the apical plasma membrane of mammary epithelial cells. This milk fat globule membrane (MFGM) can be readily obtained in quantity, and MFGM from bovine milk exhibits a higher degree of purity with respect to apical surface origin of the membrane material. Thus MFGM is an excellent source material for isolation of glycoproteins associated with the cell surface or, more properly, with a derivative of the apical cell surface. Over the past twenty years, there have been numerous publications elucidating both the isolation procedures for and the characterization of glycoproteins from milk.

With bovine MFGM, the presence of five to eight major glycoproteins have been detected with sodium dodecyl sulfate polyacrylamide gel electrophoresis (SDS-PAGE). The apparent molecular weight of these glycoproteins can range from about 30,000 to 40,000 daltons up to about 250,000 daltons. The more abundant glycoproteins have apparent molecular weights in the 30,000 to 40,000 dalton range. A mucoprotein has also been isolated from bovine milk and was found to have an apparent molecular weight of about 100,000 to 250,000 daltons.

Milk, both human and animal, natively contains an abundance of proteins including many glycoproteins. Dairy whey, a byproduct of cheese manufacturing, provides a plentiful and very inexpensive source of proteins. In fact, diary whey and products derived from dairy whey have long been recognized as food products, food additives and nutritional supplements.

Whey proteins comprise one of the two major protein groups of bovine milk, the other group being the caseins. Caseins account for about 80% of the total protein in bovine milk, while whey proteins account for the remaining approximately 20%. Whey is derived as a natural byproduct of the cheese-making process. In addition to proteins, the raw form contains fat, lactose and other substances. The raw form is processed to produce protein-rich whey protein concentrates (WPC) and whey protein isolates (WPI) among other things.

Whey proteins are comprised of high-biological-value proteins with different functions. The main whey proteins are beta-lactoglobulin and alpha-lactalbumin, two small globular proteins that account for about 70 to 80% of total whey protein. Proteins present in lesser amounts include the immunoglobulins IgG, IgA and IgM, but especially IgG, glycomacropeptides, bovine serum albumin, lactoferrin, lactoperoxidase and lysozyme. Whey proteins also contain smaller peptides derived from various proteins that are called biopeptides. Whey protein isolates are low in fat and lactose.

There are various processes for preparing whey protein isolates. Ion-exchange derived whey protein isolates are high in protein but low in glycomacropeptides, lactoferrin, lactoperoxidase and some bioactive peptides. Microfiltration/ultrafiltration derived whey protein isolates have higher amounts of glycomacropeptides, lactoferrin, lactoperoxidase and the bioactive peptides, but are lower in bovine serum albumin. Interestingly, bovine serum albumin, along with beta-lactoglobulin and IgGI, are proteins with abundant glutamylcysteine sequences. Glutamylcysteine is the precursor to glutathione. Cross-flow microfiltration gives a whey protein isolate that is greater than 90% in undenatured protein, and that retains all important sub-fractions in natural ratios, with no fat or lactose.

Recovery and purification of glycoproteins can be carried out utilizing standard methods known in the art. These would include, but are not limited to, membrane filtration and microfiltration, tangential flow filtration, chromatography (e.g., size exclusion, ion exchange, affinity), extraction, adsorption, precipitation (with non-solvents, salts, etc.), density gradient fractionation, electrophoresis, electrodialysis, isoelectric focusing, acid or base hydrolysis, and chymosin hydrolysis.

The process of isolating and recovering the glycoproteins of this invention include a heat treatment step and a flocculation step specifically designed to remove the following components of milk and milk byproducts: Lactoferrin; Immunoglobulin; Beta-lactoglobulin; Alpha-lactalbumin; and Bovine serum albumin.

Lactoferrin is a member of the Transferrin family of proteins. It is an iron binding glycoprotein isolated from milk and whey. It exerts its effect of tightly binding and metabolizing iron on the surface of glandular epithelia, secretions and mucosal surfaces as well as the interstitium and vascular compartments. It in vivo role includes disease defense and modulation of inflammatory and immune responses.

Immunoglobulin is the general name of the molecules that make up various classes of antibodies, such as IgG, IgM etc.

β-Lactoglobulin is the major whey protein in the milk of ruminants and some nonruminants, such as pigs and horses. Although beta-lactoglobulin was first isolated 60 yr ago, no function has been definitely ascribed to beta-lactoglobulin. Recent x-ray crystallographic studies have advanced knowledge of the structure of beta-lactoglobulin, which is homologous with that of retinol-binding protein and lipocalycins; the function of these proteins seems to be participation in the transport of small hydrophobic substances. By analogy, this protein has been suggested as having a role as a transporter of fatty acids and retinol. This review reassesses the function of beta-lactoglobulin in light of the large amount of information that has accrued in the last few years. In particular, this review concentrates upon studies of the binding of retinol and fatty acids to beta-lactoglobulin, including the binding constants and number of binding sites, the location of the binding sites, and the influence of chemical modifications in the interaction of the protein with both ligands. This study also describes studies of the influence of beta-lactoglobulin on several biological processes that may be relevant to the possible biological role of this protein.

α-Lactalbumin is an abundant milk-specific calcium metalloprotein which has an evolutionary relationship to lysozyme. It modifies the substrate specificity of a Golgi galactosyltransferase by forming the lactose synthetase binary complex. Lactose, together with other sugars and diffusible ions, is responsible for the osmotic pressure of milk. To assess the involvement of alpha-lactalbumin in lactogenesis, alpha-lactalbumin-deficient mice were created by disrupting the gene by homologous recombination in embryonic stem cells. Homozygous mutant mice are viable and fertile but females cannot feed their offspring. They produce a highly viscous milk that pups appear to be unable to remove from the mammary gland. This milk is rich in fat and protein and is devoid of alpha-lactalbumin and lactose. The phenotype of heterozygous mice was found to be intermediate, with a 40% decrease in alpha-lactalbumin but only a 10-20% decrease in the lactose content of their milk compared with wild-type animals. These results emphasize the key function of alpha-lactalbumin in lactogenesis and open new opportunities to manipulate milk composition.

Bovine serum albumin comes from the serum; it is not synthesized in the mammary gland. It is presumed to enter the milk via “leakage” by the paracellular pathway, or by uptake with other components such as immunoglobulins. There does not seem to be a more specific mechanism of transport for serum albumin through the secretory cell. Increases in milk concentrations of serum albumin occur especially during mastitis and during mammary gland involution. The function of serum albumin in milk is unknown. It does bind to fatty acids, as well as other small molecules.

All of the above listed milk proteins are specifically removed during the process of recovering the glycoproteins of this invention to prevent the possibility of induced sensitivity. An individual using an ophthalmic product containing any of the above listed proteins may experience an “allergic” or “immune” response. The glycoproteins of this invention are therefore considered “biocompatible” and will be safe to use in dry eye preparations

The glycoproteins of this invention are mucin-like substances that can vary in molecular weight from a few thousand to one or two hundred thousand. The glycoproteins of this invention contain oligosaccharides to the extent of about 4% to about 15%. The glycoproteins of this invention are robust and can be autoclaved without significant degradation or chemical modification.

The scientific literature reveals a number of techniques for characterizing the various types of glycoproteins. These techniques include, but are not limited to, chromatographic techniques or one or two dimensional gel electrophoresis, particularly SDS-PAGE, followed by direct protein staining (e.g., silver staining) or immunohistochemical staining (e.g., Western blotting or Northern blotting) and image analysis, immunoprecipitation techniques, amino acid analysis, carbohydrate determination, lectin binding probes, light scattering, scanning electron microscopy, mass spectrometry, peptide sequencing, MALDI, protein nitrogen content and ash.

When isolated from milk fat globules, the glycoproteins may exist as a complex. This complex may contain other components such as lipids, phospholipids, lipoproteins and oligosaccharides. These complexes can have an apparent molecular weight of from about 200,000 to 500,000 daltons or greater. In the practice of this invention either the glycoproteins themselves or the complex of glycoproteins can be utilized.

Although not being held to any one theory we believe that the glycoproteins described in this invention act to protect and lubricate the ocular surface, as in the role of the natural surface glycoproteins and mucins, which are expressed by the entire surface epithelium of the conjunctiva and cornea. By supplementing the natural epithelial surface glycoproteins, the lubrication and protection of the ocular surface is enhanced, in order to slow the progression, and associated development of symptoms, of changes to the ocular surface epithelium, such as decreased tear film stability, increased staining with fluorescein sodium or rose bengal, decreased goblet cell density and the development of squamous metaplasia seen with ocular surface disease. The property of viscosity in the preferred embodiment is primarily targeted to assist in retention of the invention in the eye at the ocular surface, as well as for lubrication and comfort associated with instillation. Viscosity is not the physical property which gives the glycoprotein formulation of this invention its “mucomimetic” function. This invention primarily protects and lubricates the ocular surface and interacts with the gel-forming secreted mucins of the tear film, thereby enhancing the spreading of the tear film, and by default of instillation adds to the tear film volume and hydration of the ocular surface. The “mucomimetic” effects of this invention protect the ocular surface from dryness and absorb shear forces of the blink, and assist the eye's own secreted gel forming mucins (predominantly MUC5) in maintaining their viscoelastic properties and ensuing structure and stability of the tear film, thereby slowing or preventing the changes to the ocular surface seen in dry eye conditions.

The amount of glycoprotein in an ophthalmic formulation can vary greatly depending on the product type. For example, in contact lens related solutions the glycoprotein concentration would vary from about 0.0001% to 5.0% by weight. In dry eye preparations the glycoprotein level could vary from about 0.1% to about 10.0% by weight. In a solid ocular insert delivery device the glycoprotein level could range to about 90% or greater by weight. Within each type of preparation the concentration might be varied, depending on such factors as the severity of the dry eye condition being treated, to enhance particular properties of the glycoprotein solution. These ranges are for the purpose of illustration and are not meant in any manner to limit the scope of the claims.

Exemplary ophthalmic compositions include a glycoprotein from any number of the exemplary sources described herein before. In addition, other formulation components may be employed as required. Examples of such formulation components include, but are not limited to:

Viscosifiers

Cellulose derivatives are commonly used to increase viscosity. Specific cellulose derivatives include: hydroxypropylmethylcellulose, carboxymethylcellulose, methylcellulose, hydroxyethylcellulose, etc. Some polysaccharides may also be utilized to increase the viscosity of ophthalmic solutions and include xanthan, scleroglucan, carrageenans, tragacanth gum, hyaluronic acid etc. Other viscosifiers that are useful include polyvinylpyrrolidone, polyvinyl alcohol, polyethyleneoxide, polyacrylic acid and crosslinked polyacrylic acid. Generally, viscosifiers are present in the amount of 0.1 to 0.75% by weight of the solution.

Buffering Agents

Any pharmaceutically acceptable buffer system may be utilized and include phosphates, borates, citrates, acetates and carbonates in amounts necessary to produce a pH of about 6.0 to about 8.0.

Tonicity Agents

The tonicity of the ophthalmic solutions described here can adjusted to either hypotonic, isotonic or hypertonic relative to normal tears by use of generally used materials know to the art. Sodium and potassium chloride are widely used to adjust tonicity. Other agents include dextrose, mannitol, sorbitol and urea.

Humectants

Water binding compounds aid in retaining moisture on the ocular surface and include glycerin, propylene glycol, polyethylene glycol.

Wetting Agents

Certain compounds are useful to promote surface wetting, whether it be the ocular surface or the surface of a contact lens. One category that is preferred is the polyoxamers. These polyethyleneoxide-polypropyleneoxide-polyethyleneoxide block copolymer are available from BASF. Other compounds include the Tetronics®, reverse Pluronics® and the reverse Tetronics®, also available from BASF.

Preservatives

The compositions of this invention may include a preservative in an effective amount. Preservatives known to the art include alkyldimethyl benzylammonium chloride (BAK), chlorhexidene gluconate (CHG), polyhexamethylene biguanide (PHMB), other polyquats and sorbic acid. The subject compositions may also include a co-preservative and/or chelating agent, such as ethylenediaminetetraacetic acid (EDTA) and its salts.

Other Additives

In some cases it may be beneficial to include other components in an ophthalmic solution. These include specific ions, such as Ca++, Zn++ and Mg++, Cu++, selenium, vitamins, such A, C and E, to promote ocular health. The compositions described in this invention may also be utilized as vehicles for drug delivery. Drugs often used in the eye include anti-glaucoma compounds, anti-inflammatory agents and anti-infective agents.

As previously described, this invention finds particular utility as lubricating eye drops, i.e., an artificial tear solution, a tear fluid supplement, a delivery vehicle for topical ophthalmic drug application. In most of these applications, the compositions of this invention are provided in a buffered, sterile aqueous solution. Typically, these solutions have a viscosity from about 1 to 100 cps. As a solution the compositions of this invention are dispensed in the eye in the form of an eye drop. It should be understood, however, that the compositions described in this invention may also be formulated as viscous liquids, i.e., viscosities from several hundred to several thousand cps, gels or ointments. In these applications the glycoprotein component could be, for example, dispersed or dissolved in an appropriate vehicle such as Lubragel, GRR Lubricating Jelly or Karajel, all trademarked products of United-Guardian, Inc., Hauppauge, N.Y.

The compositions of this invention may also be formulated as solid ocular inserts that dissolve or erode over time when placed in the cul-de-sac of the eye.

Swelling-controlled release devices would consist of glycoprotein homogeneously dispersed in a polymer such as a water soluble cellulosic. When the insert is placed in the eye, the tear fluid begins to penetrate the matrix, followed by swelling, and finally dissolution, of the matrix. As this process occurs, glycoprotein is released into the eye to provide relief of dry eye symptoms over a long period of time.

Erodible devices would again consist of glycoprotein homogeneously dispersed in a polymer matrix. In this case, glycoprotein is released by a chemical reaction (hydrolysis) that results in solubilization of the matrix polymer, usually at the surface of the device. Generally, the matrix material is a polyanhydride, poly(ortho ester), polylactic acid or a polyglycolic acid.

In another embodiment the glycoprotein may be chemically modified or crosslinked to act as its own “matrix”, where the glycoprotein comprises the entire, or nearly entire, device, thus providing the maximum amount of glycoprotein available to the eye.

Furthermore, in some contact lens related embodiments, the glycoprotein disclosed herein may be incorporated into contact lens soaking and conditioning solutions as well as lubricating eye drops for contact lens wearers.

In another embodiment the glycoproteins may be utilized in drug delivery. The most common and convenient method for delivery of ocular drugs is by way of topical eye drops. Generally, the solution vehicles employed are quickly diluted by the tear fluid and drain from the eye in a matter of minutes. This short residence time hinders the absorption and hence the bioavailability of the drug in the eye. Oftentimes the short residence time is overcome by greatly increasing the concentration of the drug to improve bioavailability. This often leads to significant undesirable side effects due to the systemic actions of many of the ocular drugs currently prescribed.

Much research has been done to improve the residence time of the drug vehicle at the ocular surface and also to promote interaction or association of the drug with the vehicle. One approach that has been commercialized is to utilize a crosslinked carboxy-functional polymer such as Carbopol®, supplied by B.F. Goodrich. The bioadhesive nature of this polymer has been the basis for controlled release ophthalmic formulations as described in U.S. Pat. No. 4,615,697 and U.S. Pat. No. 5,188,826, both of which are incorporated by reference in their entirety. These crosslinked carboxy-functional polymers swell in aqueous solution but remain as micron-size hydrated particles. Furthermore, at neutral pH, they are substantially anionic in nature. Since many ophthalmic drugs, for example timolol and pilocarpine, are positively charged, they will associate with the negatively charged polymer particles through electrostatic interaction. Also, since the hydrated particles are microporous, the drug can be absorbed into the matrix. When an ophthalmic solution of this type is placed in the eye, the hydrated polymer particles adhere to the mucosal surface, providing extended residency time. During this residence the drug is released from the hydrated polymer particles, thus providing for a more efficient local delivery to the eye.

The glycoproteins of the present invention are considered “bioadhesive” given their functions in association with the plasma membrane of mammary epithelial cells. Given this information one would expect the glycoproteins of this invention to act in a similar manner to the crosslinked carboxy-functional polymers as an ophthalmic drug delivery vehicle. In practice, the glycoproteins of this invention provide superior retention time due to their ability to interact not only with the epithelial surface but also with the natural mucins in the tear film.

The present invention provides an ophthalmic preparation for the treatment of an ocular condition known as dry eye. As such, the present invention may be described in certain embodiments as a method of treating dry eye disorders in a mammal comprising administering to said mammal an amount of glycoprotein compound effective to provide a therapeutic effect to said mammal. An aspect of the present invention is also a method of providing continued therapy to a mammal by administrating in a prescribed, regular basis to said mammal. In certain preferred embodiments of the invention, a mammal or patient to receive the glycoprotein compound may be a human or animal. Furthermore, a glycoprotein compound as used in the practice of the disclosed ophthalmic preparations and methods is glycoprotein that is derived from milk or milk byproducts. As disclosed herein and as used in the preparations and methods of the present invention, a glycoprotein compound may be formulated into a solution, ointment, gel, lotion or solid.

Effective dosages as described herein include, but are not limited to, an amount of glycoprotein compound from about 0.01 mg to about 5 mg per dose when delivered in the form of an ophthalmic solution. When the ophthalmic vehicle is a gel or ointment the amount of glycoprotein delivered can range up to about 20 mg per dose. If the glycoprotein is in the form of an ocular insert the amount of glycoprotein introduced into the eye can be up to about 150 mg. It should be noted that in the case of an ocular insert the glycoprotein is delivered by continuous release over a prolonged period of time. It is well known in the pharmaceutical art to prescribe drugs based on whether the patient is a human or animal and based on the type and severity of the disease. The ophthalmic preparations of this invention are well within the skill of a practitioner in the art. In an alternate method of describing an effective dose, an effective amount may be described, in certain embodiments as an amount that is effective to achieve a reduction in the signs and/or symptoms of dry eye in a patient.

Preferred formulations include ophthalmic compositions in which a glycoprotein is formulated as an ocular insert. More preferably, the glycoprotein is formulated as an ointment, lotion or gel. Most preferably, the glycoprotein is formulated as an ophthalmic solution.

In certain aspects, the present invention includes pharmaceutical compositions in both unit dose form and in multi-dose form. These compositions are utilized to treat dry eye, which comprises: an amount of glycoprotein such that one or more doses thereof are effective to stabilize or lessen the signs and symptoms of dry eye of said patient upon periodic administration.

Also in certain aspects, the present invention includes veterinary compositions in both unit dose form or in multi-dose form. These compositions are utilized to treat dry eye in an animal, which comprises: an amount of a glycoprotein such that one or more doses thereof are effective to stabilize or lessen the signs and symptoms of dry eye of said animal upon periodic administration.

An aspect of the present invention may also be described as a therapeutic package for dispensing to, or for use in dispensing to, a mammal being treated for dry eye syndrome comprising: one or more dosages, each dose delivered from a unit dose container or from a multi-dose container. The dosage form contains the glycoprotein of this invention, such that said one or more doses thereof are effective to stabilize or lessen the signs and symptoms of dry eye of said mammal upon periodic administration and the doses being administered periodically, and a finished pharmaceutical container or package therefor, said container containing said unit doses or multi-doses and labeling directing the use of said package in the treatment of said mammal.

The ophthalmic therapeutic preparations of this invention, in the form of a solution, lotion, ointment or gel, can be packaged in unit doses or multi-dose containers. The patient would utilize the packaged product in accordance with the prescribed regimen. Typically, in the case of an ophthalmic solution product, the patient will instill one or more drops into the eye as prescribed and/or as needed. The product container and associated packaging will bear identification, information and instructions in accordance with local, federal and foreign governmental regulations. The inclusion of a “package insert” is also generally required. The “package insert” will provide information pertaining to contents, action, indications, contraindications, warnings, how supplied, safety information and precautions, as well as directions for use.

The following examples are presented for purposes of illustrating the practice of the invention and are not intended as limitations on the scope thereof.

### EXAMPLE 1

Whey is the by product of cheese manufacturing which begins with milk and can be divided into two processes, where the major one involves enzyme treatment of milk and the minor one involves bacteria treatment.

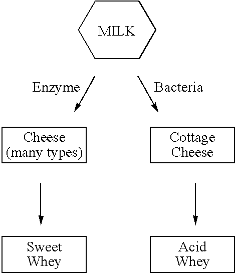

Sweet whey accounts for about 80% of whey produced and is an inexpensive and readily available source of glycoproteins.

### EXAMPLE 2

This example outlines a process for recovering the glycoproteins of this invention from sweet dairy whey. It should be noted that other processing steps could be utilized in the recovery of the glycoproteins of this invention and this example is not meant to be limiting.

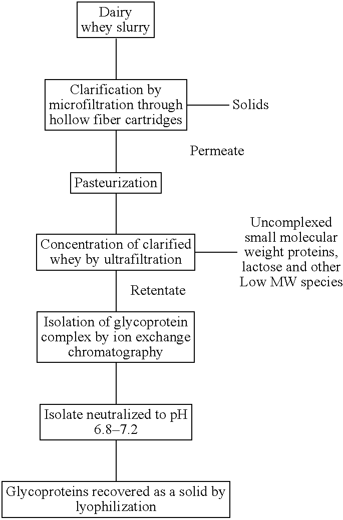

### EXAMPLE 3

The following table describes the physical and chemical properties of the glycoproteins derived from dairy whey.

Composition

| | Lot 1 | Lot 2 | Lot 3 | Lot 4 | Lot 5 | Lot 6 |
|---|---|---|---|---|---|---|
| Protein (%) | 67.0 | 60.7 | 61.4 | 67.4 | 66.4 | 66.3 |
| Lipid (%) | 21.0 | 22.2 | 25.6 | 19.4 | 22.1 | 19.7 |
| Ash (%) | 3.5 | 4.3 | 3.5 | 3.7 | 3.8 | 3.6 |
| Moisture (%) | 5.9 | 7.8 | 1.9 | 2.2 | 2.2 | 4.1 |
| pH | — | 6.7 | 6.9 | 6.9 | 6.9 | 6.9 |

Notes: Above in percent by weight. Protein determined by Leco combustion. Lipid determined by Mojonnier method. Moisture determined by weight loss under heat and vacuum. Ash determined by AOAC ignition test. pH determined on 1% concentration in phosphate buffer solution.

The above described glycoproteins are referred to as “Milcin®”, a trademark of Vista Scientific.

### EXAMPLE 4

The glycoproteins described in Example 3 are complexes composed essentially of the protein with physically associated lipids. This complex exists in aqueous solution as a hydrocolloid. A 1% by weight of Milcin® Lot 4 (Example 3) dispersion was prepared in borate buffer, pH 7.2 and osmolality of 300. Particle size of the Milcin® hydrocolloid was then determined by dynamic light scattering utilizing a Protein Solutions DynaPro MS/X instrument equipped with a Peltier temperature control device. The resulting data was analyzed with the Dynamics version 5.26.38 software provided by the manufacturer. Results were obtained using the regularization method at a grid size of 100. The following histogram was generated by the data.

[image]

[image]

From the results it can be seen that the bulk of the Milcin® hydrocolloid has a radius of about 40 nm to about 60 nm. There is a small amount of material that has a hydrodynamic radius about 100 nm.

### EXAMPLE 5

SDS-PAGE was utilized to separate the glycoproteins contained in the Milcin® complex as a function of molecular weight. This was accomplished by rendering a negative charge in the proteins through their interactions with sodium dodecylsulfate (SDS). When placed in the gel and an electric field applied, the proteins migrate as a function of their charge to molecular weight. By providing all the proteins with the same charge with SDS the proteins migrate through the gel as a function of molecular weight only, and became distributed in the bands according to molecular weight. Known amounts of protein molecular weight standards were run concurrently to demonstrate molecular weight position. The resulting gels were first oxidized then stained with a Schiff reagent to develop only the glycoprotein bands (stains glycoproteins pink). After removing the pink stain, the gel is restained with coomassie blue stain. The coomassie blue reagent reveals only the protein bands (stans proteins blue). In this manner both the glycoprotein and other proteins that are present in the Milcin® material are identified. The following diagram presents the molecular analysis of a number of Milcin® samples (see Example 3). It can be seen that the lots of Milcin®, are for the most part, composed of glycosylated protein. The molecular weight of these glycosylated proteins range from about 2000 daltons to about 200,000 daltons. The bulk of the Milcin® composition appears to be glycoprotein in the molecular weight range of about 20,000 to 100,000 daltons.

[image]

[image]

### EXAMPLE 6

The following example illustrates a method for extracting the lipids from Milcin®. A sample of Milcin® Lot 4 (see example 3) was rigorously extracted in the following manner. Approximately 1.0 gm of Milcin® (Example 3 Lot 4) was extracted with methanol for one hour and the solids filtered. The solids were then resuspended in 15 ml of chloroform and extracted for one hour and the solids filtered. The solids were then resuspended in 1 5ml of hexanes and extracted for one hour. The solids were filtered and dried in vacuum for one hour. The resulting sample was lipid free.

### EXAMPLE 7

Glycoproteins are proteins, or protein fragments, that contain carbohydrate side groups. The amount of carbohydrate can vary widely from 10% or less to over 50% by weight of the total glycoprotein. Glycoproteins typically contain N-linked and O-linked oligosaccharides. This example presents the analysis of the extracted Milcin® of Example 6 for both neutral monosaccharides and sialic acid content. A sample of the lipid extracted Milcin® of Example 6 was submitted to: Glyco Solutions Corp, 25 Winthrop St., Worcester, Mass., for analyses. The results are presented below.

Neutral Monosaccharide Analysis (pmol/8 μg Sample)

| | |
|---|---|
| Fucose | BLQ* (80 pmol) |
| GAlNac | 775 pmol |
| GlcNAc | 573 pmol |
| Galactose | 1183 pmol |
| Mannose | 696 pmol |

Below the Limit of Qualification

From the above values it is calculated that there was 0.636 μg carbohydrate 8 μg sample, or 8% neutral carbohydrate. This calculation for total carbohydrate excludes any charged monosaccharides (sialic acids, for example) that are not measured by this analysis.

Sialic Acid Analysis (pmol/8 μg** Sample)

| | |
|---|---|
| NeuAc | 1100 pmol |
| NeuGc | 16.5 pmol |

** Values were normalized to 8 μg of sample to make it consistent with previous monosaccharide composition values.

The NeuAc value was measured from the injection of 12.5% of the hydrolysate and the NeuGc value was measured from the injection of 25% of the hydrolysate. From the above values it was calculated that there was 0.346 μg sialic acid/8 μg sample, or 4.3% sialic acid.

From the above data the sample (lipid free Milcin®) contains approximately 12 to 13% by weight total carbohydrate. The results depend on the assumption that all of the carbohydrate has been hydrolyzed. If this is not the case then the true carbohydrate content will be higher.

### EXAMPLE 8

Milk products generally contain lactose unless it is specifically removed. This example presents data on the content of lactose in the Milcin® glycoprotein complex. A sample of the lipid extracted Milcin® of Example 6 was analyzed for lactose content by: Glyco Solutions Corp, 25 Winthrop St., Worcester, Mass.

The analysis revealed that the Milcin® sample contained no lactose (less than 34 ug of lactose per 100 mg of sample). This indicates that the process for recovering Milcin® glycoprotein complex from dairy whey effectively excludes lactose as an impurity.

### EXAMPLE 9

The glycoproteins of this invention as isolated from dairy whey contain a small amount of lipids that are complexed with the protein. The following example illustrates the extraction of lipids from Milcin® and the subsequent identification of those extracted lipids by liquid chromatography (TLC).

Silica gel plates were activated at 100° C. for 30 minutes and kept in a vacuum dessicator. Chromatograms were developed using various hexanes/diethyl ether solvent systems and visualized by staining with a saturated ethanolic phosphmolybdic acid solution. The lipid fraction was probed for each class of lipids by comparing the R_{f} values with control lipids in an appropriately chosen solvent system. The table below summarizes the results of the extractions and chromatograms. Each lot (see Example 3) was successively extracted three times with methanol. For the mass determination, all the methanol fractions were combined, solvent removed in vacuo and the residue was weighed.

| | Milcin ® Lot 1 | Milcin ® Lot 2 |
|---|---|---|
| Sample Weight | 995 mg | 1020 mg |
| Glycoprotein | 781 mg | 800 mg |
| Lipids | 108 mg | 173 mg |
| TLC Results | MeOH: alkyl esters, triglycerides, | MeOH: alkyl esters, triglycerides, |
| | fatty acids and polar esters. | fatty acids and polar esters |

As the results indicate Milcin® contains about 10 to 20% by weight of loosely bound lipids, that is, those extractable by methanol. These lipids are primarily alkyl esters, fatty acids, polar esters and triglycerides.

### EXAMPLE 10

The lipids extracted from Milcin® in Example 6 were subjected to analysis by gas chromatography. A series of lipids, Tripalmitate, Phosphotidylcholine dimyristate, Cholesterol oleate and Arachidic acid in a ratio of 1:10:3:30, [based on fatty acid content], were subjected to transmethylation. The resulting fatty acid methyl esters were run on High Resolution Gas Chromatography and yielded the FAME standards shown below. The retention time for each of the peaks on the H-P 1 column is proportional to the molecular weight of the FAME. Integration of these peaks showed a quantitative recovery of the starting lipids as their fatty acid methyl esters. A similar extract and transesterification of the lipids recovered in Example 6 gave the FAME profile shown below. In the extracted lipids from Example 6, we can identify myristate, palmitate and oleate. No arachidate was found.

[image]

### EXAMPLE 11

The glycoproteins of this invention would be expected to contain common metal ions. To verify the presence of these metal ions, direct current plasma (DCP) emission spectroscopy was utilized. The technique is essentially an atomic absorption technique where the spectral emission is formed in a plasma between the anode and cathode of a coupled electrode. The technique is quantitative. Milcin® Lot 4 (see Example 3) was dissolved in de-ionized water at a concentration of 1,000 ppm. Nitric acid was added (1 ppm). Proper DCP procedure was followed, including a two-point calibration (10 ppm, 0 ppm) for multi-element analyses. The results are tabulated below and reflect the averaging of six measurements for each metal.

| | Measured Concentration* | Percent by Mass in Pure |
|---|---|---|
| Metal | (ppm) | Milcin ® |
| Magnesium | 0.67 ± 0.02 | 0.067% (670 ppm) |
| Potassium | 1.8 ± 0.1 | 0.18% (1,800 ppm) |
| Sodium | 8.6 ± 0.3 | 0.86% (8,600 ppm) |
| Copper | 0.06 ± 0.20 | 0.006% (60 ppm) |
| Zinc | 0.2 ± 0.2 | — |

the measured concentration is the concentration of the metal in a 1,000 ppm solution of pure Milcin® Lot 4.

### EXAMPLE 12

The following example illustrates the use of the glycoproteins of this invention as the active ingredients in an ophthalmic solution for the treatment of dry eye signs and symptoms. The formulation listed below:

| INGREDIENTS | AMOUNT IN GRAMS |
|---|---|
| Milcin ® Lot 4 | 1.05 |
| Hydroxyethylcellulose Natrosol 250 M Pharm | 0.4 |
| Sodium chloride | 0.48 |
| Sodium borate decahydrate | 0.12 |
| Boric acid | 0.74 |
| Water | 97.21 |

was prepared in the following manner:

1. Weigh out the water and add to an appropriate beaker equipped with a stirring apparatus.

2. With stirring, add the sodium chloride, sodium borate decahydrate and boric acid to the water.

3. Stir until the salts have completely dissolved, about 10 minutes.

4. With vigorous stirring, add the Milcin® slowly to the batch.

5. Continue vigorous stirring until the Milcin® is finely dispersed, about 30 minutes.

6. With moderate stirring, add the hydroxyethylcellulose slowly to the batch.

7. Continue stirring at a moderate rate for 2 hours.

8. Allow the batch to deaerate for 15 minutes.

9. Place batch in a suitable sealed container for autoclaving.

10. Autoclave the batch for 60 minutes at 121° C.

11. Remove solution container from the autoclave.

12. Open container and perform batch testing.

13. Once prepared, the formulation listed above was tested and the following solution properties were determined.

| PROPERTY | VALUE |
|---|---|
| Viscosity, cps | 24 |
| Osmolality, mOsm/kg | 304 |
| pH | 7.2 |
| Appearance | Slightly hazy |
| Color | Bluish tint |

### EXAMPLE 13

This example illustrates the ocular compatibility of the glycoproteins of this invention utilizing an in vitro transepithelial permeability assay.

The irritation potential of Milcin® was evaluated at a level of 1.05% in an ophthalmic solution. The commercially available lubricant eye drop solution “Refresh Tears”, manufactured and sold by Allergan, was also evaluated.

The following solution formulations were prepared utilizing the compounding procedure detailed in Example 12. The finished solutions were transferred aseptically into sterilized bottles.

| INGREDIENT | A | B |
|---|---|---|
| Milcin ® Lot 4, gms | — | 1.05 |
| Hydroxyethylcellulose Natrosol 250 M Pharm, gms | 0.40 | 0.40 |
| Sodium chloride, gms | 0.48 | 0.48 |
| Sodium borate decahydrate, gms | 0.12 | 0.12 |
| Boric acid, gms | 0.74 | 0.74 |
| Water, gms | 98.26 | 97.21 |

The solutions described in the table above were subjected to the following experiments to determine potential eye irritation of the solutions. The experimental methods follow the procedure developed by R. Tchao, which is described in “Trans-Epithelial Permeability of Fluorescein In Vitro as an Assay to Determine Eye Irritants”, Progress in In Vitro Toxicology, Volume 6, 1988, pages 271-283 (Mary Ann Liebert, Inc. Publishers, New York), the disclosure of which is incorporated herein by reference. The Tchao technique is described as a method of determining potential eye irritation of a substance by correlating damage to a mono-layer of Madin-Darby Canine Kidney (MDCK) cells with damage to corneal epithelial cells. The amount of fluorescein passing through the cell mono-layer is a function of permeability of the cell mono-layer. Higher cell mono-layer permeability indicates greater damage to the cell junctions from application of a test solution thereto, whereas lower cell mono-layer permeability indicates less damage to the cell junctions from application of the test solution.

The details of the test are presented below.

Culture preparation: MDCK cells are obtained from ATCC, and maintained in minimum essential medium (MEM) supplemented with 10% bovine calf serum with iron supplementation (Hyclone, Utah). Stock cultures are passaged weekly using trypsin and EDTA. Cultures are used before passage 50. For the test, 0.5ml of a cell suspension containing 2×10 E5 cells are seeded in Millicell HA 13 mm inserts (Millipore, Bedford, Mass.). The inserts are placed in 24-well plates and fed with 0.5ml medium. Two days after seeding the cells, the media both inside and outside the inserts are replaced with fresh media. On day 6 after seeding, the inserts are used for testing the solutions. It has been shown that the resistance developed by a confluent MDCK monolayer is about 600 ohms/cm².

Test: Each insert is rinsed with Hanks Balanced Salt Solution (HBSS) 3×1 ml using a 10 ml syringe without needle. Each test solution (0.5 ml) is added to the inside of an insert that has been placed in a fresh 24-well plate. Triplicate inserts are used for each test solution. The 24-well plate with inserts and test solutions are placed in a humidified incubator at 37° C. for 30 minutes. Each series of triplicates is handled sequentially to allow exact timing of the treatment. After incubation, sequentially, each insert is individually rinsed with HBSS 5×1 ml using the 10 ml syringe, and is placed in a fresh 24-well plate containing 0.5 ml HBSS in each well. 0.5 ml of a solution of Na-fluorescein (3 mg/100 ml) is added to each rinsed insert. After incubation at room temperature for 30 minutes, the inserts are sequentially removed from the wells, and the amount of Na-fluorescein in each of the wells is measured in a CytoFluor 2300, using 540 nm excitation and 590 nm emission. For each test, the negative control is HBSS and the positive control is 250 μg/ml sodium dodecyl sulfate (SDS). It has been determined that the assay can measure the effect of 50 μg/ml SDS, and the effect on the permeability of the monolayer is linearly proportional to the concentrations of SDS from 50-250 μg/ml. Fluorescence units (arbitrary) of each test solution is plotted against test solutions.

Interpretation of results: The results are expressed as % of SDS response, and comparisons with the HBSS response. Generally, if the solution is 20% of the SDS response, the solution may be a mild irritant.

The results of the in vitro irritation potential testing are presented below along with the results for the positive and negative controls. The positive control 250 ppm of sodium dodecyl sulfate (SDS) is known to cause noticeable irritation when instilled in the human eye. The negative control Hank's balanced salt solution (HBSS) is known not to elicit any adverse reaction when instilled in the human eye. The results are expressed as a percentage of SDS response, that is, SDS=100% response. Any response less than 20% indicates little or no tissue change and is considered non-irritating.

| SOLUTION | RESPONSE |
|---|---|
| SDS (250 ppm) | 100 |
| Solution A | 0.64 ± 0.1 |
| Solution B | 0.86 ± 0.1 |
| Refresh Tears | 2.45 ± 0.5 |
| HBSS | 3.00 ± 0.5 |

It can be seen that the response of the Milcin® solution (B) is the same as the control solution (A) and both are well below the response of the negative control HBSS. Given this data, Milcin® based ophthalmic solutions should be completely compatible with the ocular environment.

### EXAMPLE 14

The following example illustrates the robustness of the glycoprotein of this invention with respect to autoclaving. The formulation listed below:

| INGREDIENT | AMOUNT IN GRAMS |
|---|---|
| Milcin ® Lot 4 | 1.05 |
| Hydroyethylcellulose Natrosol 250 M Pharm | 0.40 |
| Sodium chloride | 0.48 |
| Sodium borate decahydrate | 0.12 |
| Boric acid | 0.74 |
| Water | 97.21 |

was prepared by the detailed process given in Example 12 except that half the batch was autoclaved and half the batch was not (steps 1 through 8 only in Example 12).

Once prepared, both formulations representing both process conditions were tested and the following solution property was determined.

| | Before Autoclaving | After Autoclaving |
|---|---|---|
| PROPERTY | VALUE | VALUE |
| Viscosity, cps | 37 | 24 |
| Osmolality, mOsm/kg | 293 | 304 |
| pH | 7.2 | 7.2 |
| Appearance | Slightly hazy | Slightly hazy |
| Color | Bluish tint | Bluish tint |

The above results confirm the robustness of Milcin® and demonstrate the ability of Milcin® containing ophthalmic solution to be sterilized by autoclaving.

### EXAMPLE 15

The following example illustrates the use of lipid free glycoproteins in an ophthalmic solution. The extracted glycoproteins recovered in Example 6 were utilized as the active ingredients in the ophthalmic solution formulation described below:

| INGREDIENT | AMOUNT IN GRAMS |
|---|---|
| Extracted Milcin ® (see Example 6) | 1.00 |
| Hydroxyethylcellulose Natrosol 250 M Pharm | 0.50 |
| Propylene glycol | 0.50 |
| Sodium chloride | 0.20 |
| Sodium borate decahydrate | 0.12 |
| Boric acid | 0.70 |
| Potassium sorbate | 0.15 |
| Edetate disodium | 0.05 |
| Water | 96.78 |

The formulation was prepared by the detailed process given in Example 12. The finished solution was tested and the following physical properties were generated.

| PROPERTY | VALUE |
|---|---|
| Viscosity, cps | 27.0 |
| Osmolality, mOsm/kg | 317 |
| pH | 6.6 |
| Appearance | Clear |
| Color | Water white |

### EXAMPLE 16

The example illustrates the preparation of a preserved ophthalmic solution utilizing the glycoproteins of this invention.

| INGREDIENT | AMOUNT |
|---|---|
| Milcin ® Lot 6 | 3.00 gms |
| Sodium chloride | 0.48 gms |
| Sodium borate decahydrate | 0.11 gms |
| Boric acid | 0.75 gms |
| Polyhexamethylene biguanide | 10 ppm |
| Purified water | 95.66 gms |

The above formulation as prepared by dissolving the salts in water followed by the addition of the polyhexamethylene biguanide and then the Milcin®. The batch is then vigorously mixed for one hour. The resulting solution was translucent with a pH of 6.9 and an osmolality of 305 mOsm/kg.

### EXAMPLE 17

The following example illustrates the use of glycoproteins in an ophthalmic gel for the treatment of dry eye signs and symptoms. Lubragel® MS, available from United-Guardian, Inc. was chosen as the gel base. Lubragel® MS is composed of polyglycerolmethacrylate and propylene glycol preserved with parabens. A 2% Milcin® (Lot 6) in Lubragel® MS was prepared by thoroughly mixing the Milcin® into the gel base to form a uniform dispersion. The resulting gel was slightly hazy.

### EXAMPLE 18

This example illustrates the use of the glycoproteins of this invention in contact lens solutions. The contact lens solution base utilized in this example was RENU™ MultiPlus, manufactured and sold by Bausch & Lomb. RENU™ is a multi-purpose solution for the storage and care of soft hydrogel contact lens. The following formulation was prepared by adding 0.5% by weight of Milcin® to RENU™.

| INGREDIENT | AMOUNT IN GRAMS |
|---|---|
| Milcin ® Lot 6 | 0.25 |
| RENU ™ Lot CH7058 | 49.75 |

The Milcin® was thoroughly dispersed in the RENU™ by vigorous stirring for one hour. The resulting solution was slightly hazy with a pH of 7.1 and an osmolality of 292 mOsm/kg. It is expected that the addition of Milcin® to a soft contact lens solution will result in improved lubricity and comfort to the lens wearer.

### EXAMPLE 19

The following example illustrates the use of glycoproteins as an erodible ocular insert to provide long lasting treatment of dry eye. Approximately 50 mg of Milcin® Lot 3 (see Example 3) was placed in a micro KBr press. The press was heated to about 80° C. and then tightened to compress the Milcin® under heat and pressure. The KBr press was allowed to cool to room temperature and the sample was removed. The Milcin® was in the form of a solid disk about 5 mm in diameter and about 2 mm in thickness. The Milcin® disk was placed in water and slowly eroded over about two hours.

### EXAMPLE 20

This example illustrates the use of the glycoproteins of this invention as a component in an allergy relief solution. The particular ingredient for allergy relief chosen was olopatadine hydrochloride. Patanol® is a commercially available solution containing 0.1% by weight of olopatadine. The other solution components are sodium chloride, a phosphate buffer system and benzalkonium chloride as a preservative. Patanol® is an isotonic solution with a pH of about 7. A solution was prepared by adding 1.0% by weight of Milcin® Lot 6 (see Example 3) into Patanol® solution which is manufactured and sold by Alcon Pharmaceuticals. The Milcin® was compatible in the Patanol® solution and is expected to provide improved lubricity and comfort to the patient.

### EXAMPLE 21

This example illustrates the use of the glycoproteins of this invention in an antibacterial ophthalmic solution with activity against a broad spectrum of gram-postive and gram-negative ocular pathogens. The antibacterial agent chosen was ciprofloxacin hydrochloride. Ciloxan® is a commercially available solution containing 0.3% ciprofloxacin. The other solution ingredients are sodium acetate, mannitol, edetate disodium and benzalkonium chloride. Ciloxan® is an isotonic solution with a pH about 4.5 that is manufactured and sold by Alcon Pharmaceuticals. A solution was prepared by adding 1.0% by weight of Milcin® Lot 6 (see Example 3) into Ciloxan® solution. The Milcin® was compatible in the Ciloxan® solution and is expected to provide improved lubricity and comfort to the patient.

### EXAMPLE 22

This example illustrates the use of the glycoproteins of this invention in an antibiotic and steroid combination ophthalmic solution. The antibiotic agent chosen was tobramycin and the steroid chosen was dexamethasone. Tobradex® is a commercially available solution containing 0.3% by weight tobramycin and 0.1% by weight of dexamethasone. The other solution ingredients are hydroxethylcellulose, sodium chloride, sodium sulfate, Tyloxapol®, edetate disodium and benzylalkonium chloride as the preservative. Tobradex® is manufactured and sold by Alcon Pharmaceuticals. A solution was prepared by adding 1.0% by weight of Milcin® Lot 6 (see Example 3) into Tobradex® solution. The Milcin® is expected to provide improved lubricity and comfort to the patient.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing form the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An ophthalmic preparation comprising a glycoprotein derived from one of mammalian milk and a milk byproduct.

2. An ophthalmic preparation in accordance with claim 1 wherein the glycoprotein component is derived from dairy whey.

3. An ophthalmic preparation comprising a glycoprotein component substantially free of: lactoferrin; immunoglobulin; beta-lactoblobulin; alpha-lactalbumin; and bovine serum albumin.

4. An ophthalmic preparation in accordance with claim 3 wherein the glycoprotein containing components have a molecular weight of from about 3,000 daltons to about 250,000 daltons.

5. An ophthalmic preparation in accordance with claim 3 wherein the glycoprotein containing components have a carbohydrate content of about 3% by weight to about 50% by weight.

6. An ophthalmic preparation in accordance with claim 3 wherein said preparation is in the form of one of a solution, an ointment and an ocular insert.

7. An ophthalmic preparation in accordance with claim 3 wherein the glycoprotein is present in an amount from about 0.001% to about 10.0% by weight.

8. An ophthalmic preparation in accordance with claim 3 wherein the glycoprotein is present in an amount from about 10% to about 90% by weight.

9. An ophthalmic preparation in accordance with claim 1 wherein the glycoprotein is complexed with at least one component selected from the group consisting of a lipid, phospholipid and lipoprotein.

10. An ophthalmic preparation in accordance with claim 9 wherein the lipid component is present in the amount of 0.01% to about 30% of the complex.

11. An ophthalmic preparation in accordance with claim 1 wherein the glycoprotein is autoclavable.

12. An ophthalmic preparation in accordance with claim 1 further comprising a material selected from the group consisting of a buffering agent; a viscosity modifying agent; a tonicity modifying agent; a humectant compound; and a therapeutic drug.

13. An ophthalmic preparation in accordance with claim 3 wherein said glycoprotein is derived from sweet whey.

14. An ophthalmic preparation in accordance with claim 13, wherein said glycoprotein is derived from purified whey.

15. A method of treating dry eye in a mammal comprising administering an ophthalmic preparation to said mammal in need thereof an amount of a glycoprotein component that is contained therein and that is effective to treat the dry eye of said mammal, wherein the glycoprotein component is substantially free of: lactoferrin; immunoglobulin; beta-lactoblobulin; alpha-lactalbumin; and bovine serum albumin.

16. The method of claim 15, wherein said effective amount of the glycoprotein component is from about 0.01 mg to about 5.0 mg per dose.

17. The method of claim 15, wherein said effective amount of the glycoprotein component is from about 5.0 mg to about 20.0 mg per dose.

18. The method of claim 15, wherein said effective amount of the glycoprotein component is from about 20.0 mg to about 200 mg per dose.

19. A therapeutic package for dispensing to, or for use in dispensing to, a patient being treated for dry eye comprising:
one or more unit doses, each such unit dose comprising an amount of glycoprotein therein such that periodic administration of one or more of said unit doses is effective to treat said dry eye condition, and
a finished pharmaceutical container therefore, said container containing said unit dose or multiple doses, said container further including labeling;
said labeling directing the use of said package in the treatment of said dry eye condition in a dosage regimen under which the delivery of said glycoprotein is confined to the period during the day proximate to the time of day at which the patient requires treatment, and further directing the use of said package in conjunction with the concomitant administration to said patient of one or more unit doses providing a therapeutically effective amount of glycoprotein to said patient.

20. A package according to claim 19 in which said glycoprotein is substantially free of: lactoferrin; immunoglobulin; beta-lactoblobulin; alpha-lactalbumin; and bovine serum albumin.

21. A package according to claim 19 in which the delivery of said glycoprotein is directed to be confined proximate to the time of waking and the delivery of said glycoprotein is confined proximate to the time of onset of sleeping.

22. A therapeutic package for dispensing to, or for use in dispensing to, a patient being treated for dry eye comprising:
one or more unit doses, each such unit dose comprising an amount of glycoprotein therein such that periodic administration of one or more of said unit doses is effective to treat said dry eye condition, wherein said glycoprotein is substantially free of: lactoferrin; immunoglobulin; beta-lactoblobulin; alpha-lactalbumin; and bovine serum albumin; and
a finished pharmaceutical container therefore, said container containing said unit dose or multiple doses, said container further containing or comprising labeling;
said labeling directing the use of said package in the treatment of said dry eye condition in a dosage regimen under which the delivery of said glycoprotein is one or more times daily, or as directed by a physician.

23. A package according to claim 22 wherein said labeling directs the use of said package in the treatment of dry eye induced by an activity selected from the group consisting of: contact lens wear and prolonged viewing of a computer screen.

24. A package according to claim 22 that is produced by form, fill and seal manufacturing wherein each container contains from about 0.50 ml to about 1.50 ml of glycoprotein solution.

25. A package according to claim 22 comprising a bottle, dropper tip and cap wherein each bottle contains from about 2.0 ml to about 30.0 ml of glycoprotein solution.

26. A package according to claim 24 in which said unit dose containers are provided to accommodate an uninterrupted dosage regimen basis of at least one month.
